(19) United States
(12) Patent Application Publication
(10) Pub. No.: US 2018/0110402 A1
(43) Pub. Date: Apr. 26, 2018

(54) SCANNING ENDOSCOPE SYSTEM

(71) Applicant: OLYMPUS CORPORATION, Tokyo (JP)

(72) Inventors: Masashi YAMADA, Tokyo (JP); Atsuyoshi SHIMAMOTO, Tokyo (JP); Mitsuru NAMIKI, Saitama (JP); Keiichiro NAKAJIMA, Tokyo (JP); Takamitsu SAKAMOTO, Tokyo (JP); Mikihiko TERASHIMA, Tokyo (JP); Yoko OKABE, Tokyo (JP)

(73) Assignee: OLYMPUS CORPORATION, Tokyo (JP)

(21) Appl. No.: 15/839,095

(22) Filed: Dec. 12, 2017

Related U.S. Application Data
(63) Continuation of application No. PCT/JP2016/068012, filed on Jun. 16, 2016.

(30) Foreign Application Priority Data
Jun. 24, 2015 (JP) PCT/JP2015/068195
Mar. 4, 2016 (JP) 2016-042729

(51) Int. Cl. A61B 1/00; A61B 1/05; A61B 1/07

(52) U.S. Cl. CPC A61B 1/00009; A61B 1/00172; A61B 1/045; A61B 1/07; A61B 1/05

(57) ABSTRACT

Provided is a scanning endoscope system including an illumination-light emitting unit that outputs illumination light emitted from a light source in a spot-like manner toward a subject, a light scanner that scans the illumination light output from the illumination-light emitting unit over the subject, and a light detector that is provided in a movable manner relative to the illumination-light emitting unit and that detects reflection light from a scan position on the subject over which the illumination light is scanned by the light scanner.

### CROSS-REFERENCE TO RELATED APPLICATIONS

This is a continuation of International Application PCT/JP2016/068012 which is hereby incorporated by reference herein in its entirety.

This application is based on International Application PCT/JP2015/068195 and Japanese Patent Application No. 2016-042729, the contents of which are incorporated herein by reference.

### TECHNICAL FIELD

The present invention relates to scanning endoscope systems.

### BACKGROUND ART

In a known scanning endoscope in the related art, illumination light is scanned over a subject by vibrating an illumination optical fiber that optically guides the illumination light, and reflection light from the surface of the subject is received by detection optical fibers, thereby forming an image (for example, see Patent Literature 1). In this scanning endoscope, a plurality of detection optical fibers are arranged and fixed in the circumferential direction of a cylindrical scanning unit that vibrates the illumination optical fiber.

### CITATION LIST

### Patent Literature

{PTL 1}

U.S. Pat. No. 6,294,775

### SUMMARY OF INVENTION

An aspect of the present invention provides a scanning endoscope system including: an illumination-light emitting unit that outputs illumination light emitted from a light source in a spot-like manner toward a subject; a light scanner that scans the illumination light output from the illumination-light emitting unit over the subject; and a light detector that is provided in a movable manner relative to the illumination-light emitting unit and that detects reflection light from a scan position on the subject over which the illumination light is scanned by the light scanner.

### BRIEF DESCRIPTION OF DRAWINGS

FIG. 1 is a block diagram illustrating a scanning endoscope system according to an embodiment of the present invention.

FIG. 2 illustrates an example of a state where a first insertion section and a second insertion section of the scanning endoscope system in FIG. 1 are inserted in the body.

FIG. 3 is a vertical sectional view of the first insertion section of the scanning endoscope system in FIG. 1.

FIG. 4 is a side view of an optical-fiber support member of the first insertion section in FIG. 3.

FIG. 5 is a cross-sectional view of the optical-fiber support member in FIG. 4.

FIG. 6 illustrates an example of the positional relationship between the first insertion section and the second insertion section of the scanning endoscope system in FIG. 1.

FIG. 7 illustrates a case where a scan range of illumination light from the first insertion section in FIG. 6 is expanded and the second insertion section is moved away from a subject.

FIG. 8 illustrates a case where the first insertion section in FIG. 6 is moved close to the subject.

FIG. 9 illustrates a first modification of the scanning endoscope system in FIG. 1 and is a block diagram illustrating a case where a light detector is disposed at the distal end of the second insertion section

FIG. 10 illustrates an example of the light detector of the scanning endoscope system in FIG. 9.

FIG. 11 illustrates a second modification of the scanning endoscope system in FIG. 1 and is a block diagram illustrating a case where a plurality of second insertion sections are provided.

FIG. 12 illustrates an example of the light detector of the scanning endoscope system in FIG. 11.

FIG. 13 illustrates a third modification of the scanning endoscope system in FIG. 1 and is a block diagram illustrating a case where the first insertion section is also provided with a light detector.

FIG. 14 is a front view illustrating an example of the arrangement of the light detector in the first insertion section of the scanning endoscope system in FIG. 13.

FIG. 15 is a front view illustrating another example of the arrangement of the light detector in the first insertion section of the scanning endoscope system in FIG. 13.

FIG. 16 illustrates a fourth modification of the scanning endoscope system in FIG. 1 and shows an example where light from an optical fiber of the second insertion section and an optical fiber of the first insertion section are focused onto a light detector.

FIG. 17 illustrates a fifth modification of the scanning endoscope system in FIG. 1 and shows an example where the first insertion section and the second insertion section are connected to each other in a wireless manner.

### DESCRIPTION OF EMBODIMENTS

A scanning endoscope system 1 according to an embodiment of the present invention will be described below with reference to the drawings.

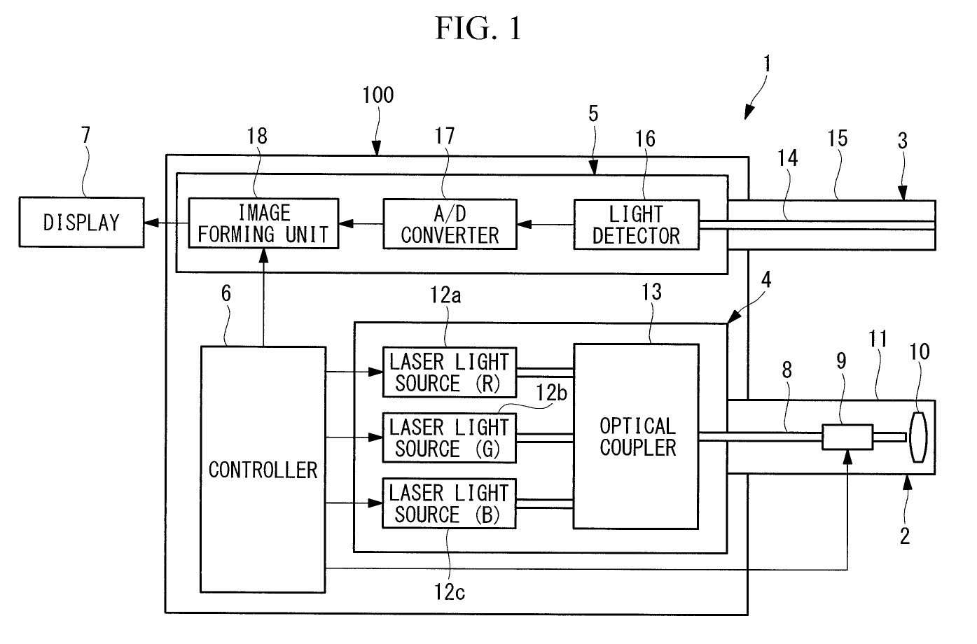

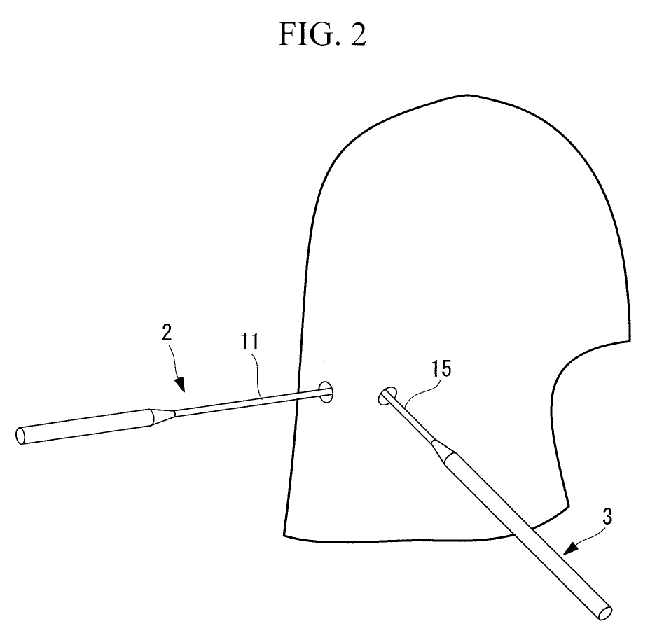

As shown in FIGS. 1 and 2, the scanning endoscope system 1 according to this embodiment includes a first insertion section (illumination-light emitting unit) 2 and a second insertion section (light detector) 3 that are to be inserted into a patient, a light source unit (light source) 4 connected to the first insertion section 2, an image acquiring unit 5 connected to the second insertion section 3, a controller 6 that controls the first insertion section 2, the light source unit 4, and the image acquiring unit 5, and a display 7 that displays an image acquired by the image acquiring unit 5.

The first insertion section 2 and the second insertion section 3 are separate components and can move relatively freely. The light source unit 4, the image acquiring unit 5, and the controller 6 are accommodated within a housing 100.

The first insertion section 2 includes an optical fiber 8 formed of a single mode fiber that is disposed at the center of the first insertion section 2 and that optically guides light from the light source unit 4, a light scanner 9 that is provided at the distal end of the optical fiber 8 and that vibrates an output end 8a of the optical fiber 8 so as to two-dimensionally scan light output from the output end 8a, an illumination lens 10 that focuses the illumination light output from the output end 8a of the optical fiber 8 so as to form a spot on a subject, and a cylindrical protection member 11 that covers the above components.

The light scanner 9 is, for example, a piezoelectric element and causes bending vibrations to occur in the optical fiber 8 in accordance with an input voltage.

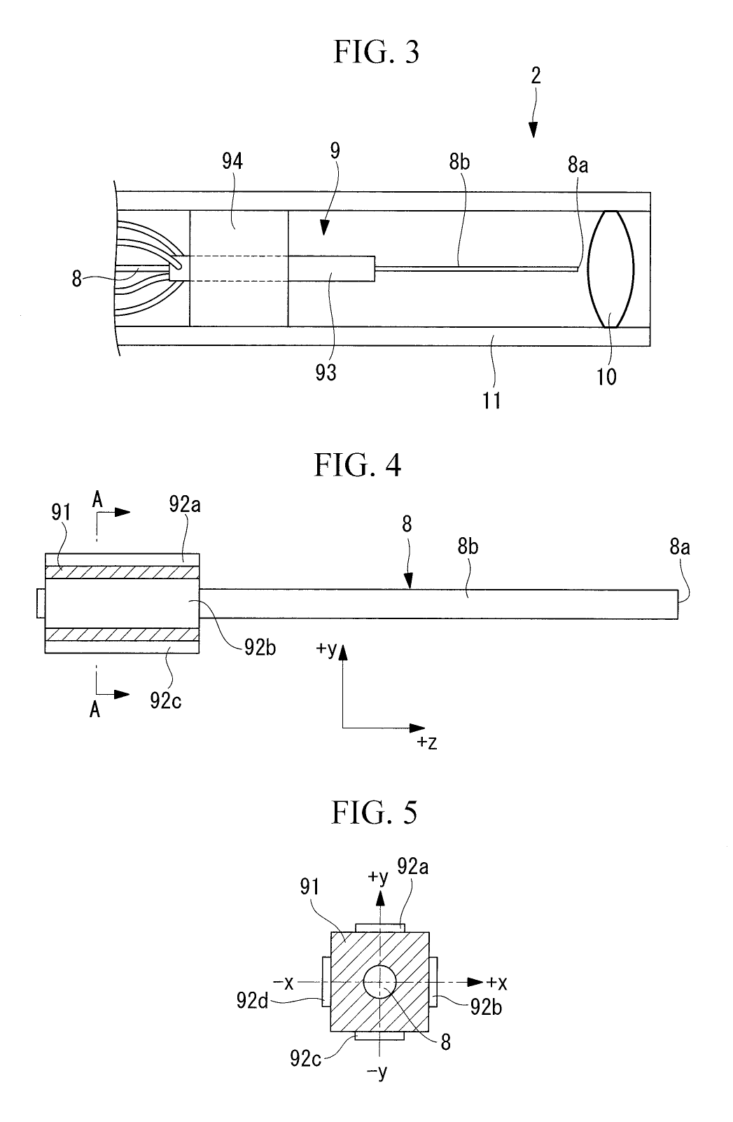

The first insertion section 2 will be described in detail with reference to FIGS. 3 to 5.

The light scanner 9 is, for example, an actuator. As shown in FIGS. 3 and 4, the light scanner 9 is constituted of vibration transmittable components and includes an optical-fiber support member 91 that supports the optical fiber 8, piezoelectric elements 92a, 92b, 92c, and 92d arranged along the outer periphery of the optical-fiber support member 91, an actuator tube 93 that covers the piezoelectric elements 92a, 92b, 92c, and 92d and the optical-fiber support member 91, and an attachment ring 94 that fixes the actuator tube 93 to the protection member 11.

As shown in FIG. 4, the optical fiber 8 is supported by the optical-fiber support member 91 and has an oscillating section 8b that extends from the optical-fiber support member 91 to the output end 8a and that is vibrated by the piezoelectric elements 92a, 92b, 92c, and 92d.

As shown in FIGS. 4 and 5, the optical-fiber support member 91 has a square pillar shape the four side surfaces of which are orthogonal to each other and are also perpendicular to the light output direction (optical axis direction) at the output end 8a of the optical fiber 8. Specifically, the four side surfaces of the optical-fiber support member 91 are perpendicular to the +z direction in FIG. 4 and are respectively oriented in the +x direction, +y direction, −x direction, and −y direction in FIG. 5 so as to be orthogonal to each other.

The pair of piezoelectric elements 92a and 92c for driving in the y direction are fixed to the +y side and the −y side of the optical-fiber support member 91, and the pair of piezoelectric elements 92b and 92d for driving in the x direction are fixed to the +x side and the −x side. Of each pair of piezoelectric elements disposed facing each other with the optical-fiber support member 91 interposed therebetween, one of the piezoelectric elements contracts when the other expands, thus causing the optical-fiber support member 91 to deform. The pairs of piezoelectric elements alternately repeat this process so as to cause vibration to occur in the x and y directions, thereby two-dimensionally scanning the output end 8a of the optical fiber 8. The illumination light output from the output end 8a of the optical fiber 8 vibrated in this manner is focused onto an observation target by the illumination lens 10.

The light source unit 4 includes three laser light sources 12a, 12b, and 12c, such as laser diodes, respectively generating red light, green light, and blue light, and also includes an optical coupler 13 that multiplexes the light beams of the three colors from the laser light sources 12a, 12b, and 12c and that optically guides the light to the optical fiber 8. The optical coupler 13 is constituted of, for example, a fiber-type combiner or a dichroic prism.

The second insertion section 3 includes an optical fiber 14 formed of a multimode fiber having a light-receiving end that receives reflection light from a subject, and also includes a cylindrical protection member 15 that covers the optical fiber 14.

The image acquiring unit 5 includes a light detector 16, such as an avalanche photodiode, which performs photoelectric conversion on the reflection light received at the light-receiving end of the optical fiber 14 and optically guided through the optical fiber 14, an analog-to-digital (A/D) converter 17 that converts an analog signal into a digital signal based on the intensity of the reflection light detected by the light detector 16, and an image forming unit 18 that forms an image based on an output from the A/D converter 17.

The controller 6 controls the lighting timings of the three laser light sources 12a, 12b, and 12c and also controls the positions to which the beams of illumination light from the laser light sources 12a, 12b, and 12c are scanned by the light scanner 9. Furthermore, the controller 6 sends, to the image forming unit 18, scan position information about the illumination light output from the first insertion section 2.

The image forming unit 18 forms an image based on the intensity information about the reflection light output from the A/D converter 17 and the scan position information about the illumination light sent from the controller 6. The image formed by the image forming unit 18 is sent to the display 7.

The operation of the scanning endoscope system 1 according to this embodiment having the above-described configuration will be described below.

In order to observe the inside of the body of a patient by using the scanning endoscope system 1 according to this embodiment, the second insertion section 3 is first inserted into the body, and the first insertion section 2 is subsequently inserted into the body.

When the first insertion section 2 is inserted into the body, the controller 6 is actuated so as to cause the three laser light sources 12a, 12b, and 12c to sequentially output three kinds of illumination light in a predetermined light emission order (e.g., in the order R, G, and B). In addition, the light scanner 9 is controlled in accordance with a command signal from the controller 6, so that the illumination-light scan positions are sequentially changed. For example, by actuating the light scanner 9, the output end of the optical fiber 8 provided in the first insertion section 2 is moved in a spiral manner, whereby the beams of illumination light are radiated such that the spots of illumination light are arranged on a spiral trajectory on the subject.

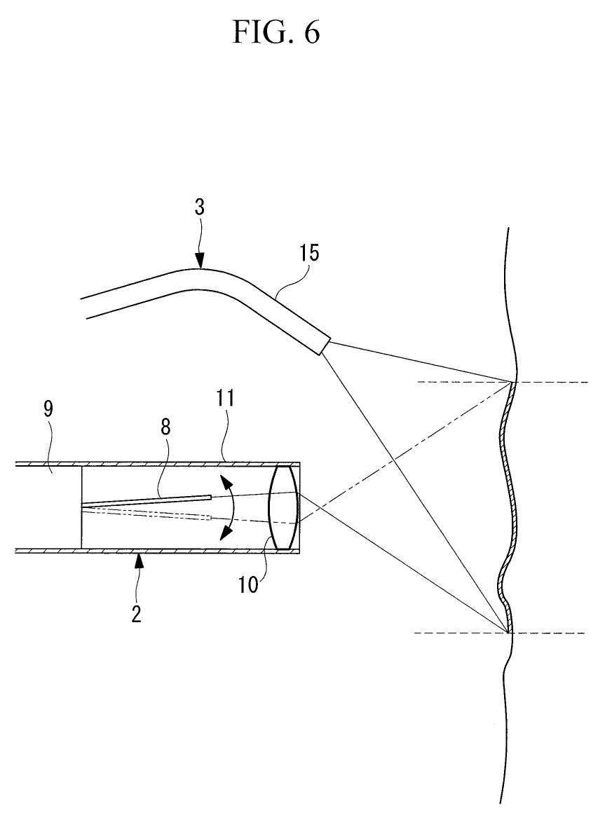

As shown in FIG. 6, when the beams of illumination light are output from the laser light sources 12a, 12b, and 12c, a portion of the reflection light at each scan position on the subject is received by the light-receiving end of the optical fiber 14 of the second insertion section 3 inserted in the body in advance, and is detected by the light detector 16. The intensity information about the reflection light detected by the light detector 16 is converted into a digital signal by the A/D converter 17 and is then sent to the image forming unit 18.

Because the image forming unit 18 receives, from the controller 6, the scan position, on the subject, of the spot of illumination light corresponding to the intensity information about the reflection light and the information about the color of the light radiated onto that position, the image forming unit 18 can generate a two-dimensional color image by arranging the color and intensity information of the detected reflection light in correspondence with the scan position.

In this case, because the first insertion section 2 and the second insertion section 3 to be inserted into the body are separated from each other and are relatively movable, the scanning endoscope system 1 according to this embodiment has the following advantages.

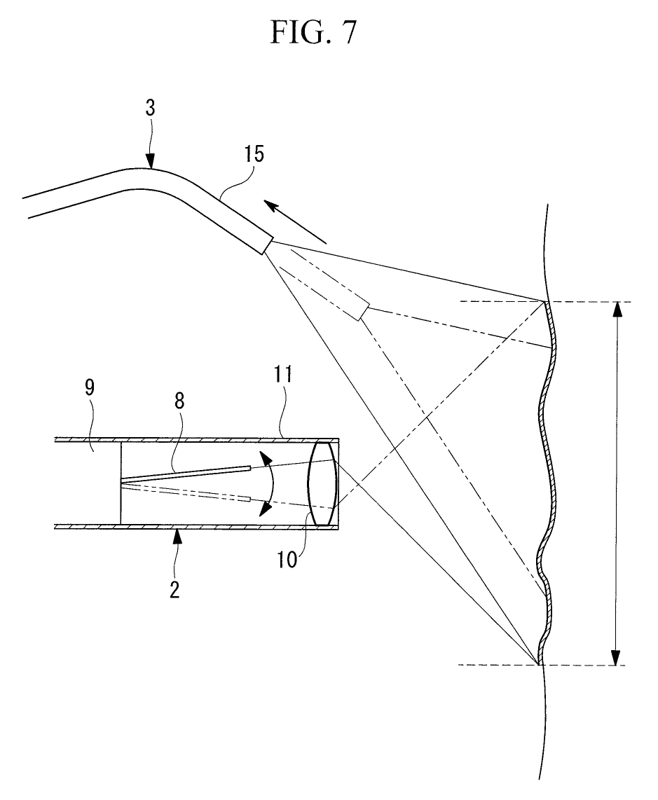

Specifically, if the illumination-light illumination range set by the light scanner 9 based on the vibration amplitude of the output end 8a of the optical fiber 8 exceeds the light-receiving range of the optical fiber 14 of the second insertion section 3, a photographic field angle that covers the illumination range can be realized by simply moving the second insertion section 3 relatively to the first insertion section 2 in the direction away from the subject, as indicated by an arrow in FIG. 7.

In this case, there is no change in the spot diameter of the illumination light in the subject since the position of the first insertion section 2 is changed. This is advantageous in that the photographic field angle can be increased without lowering the resolution.

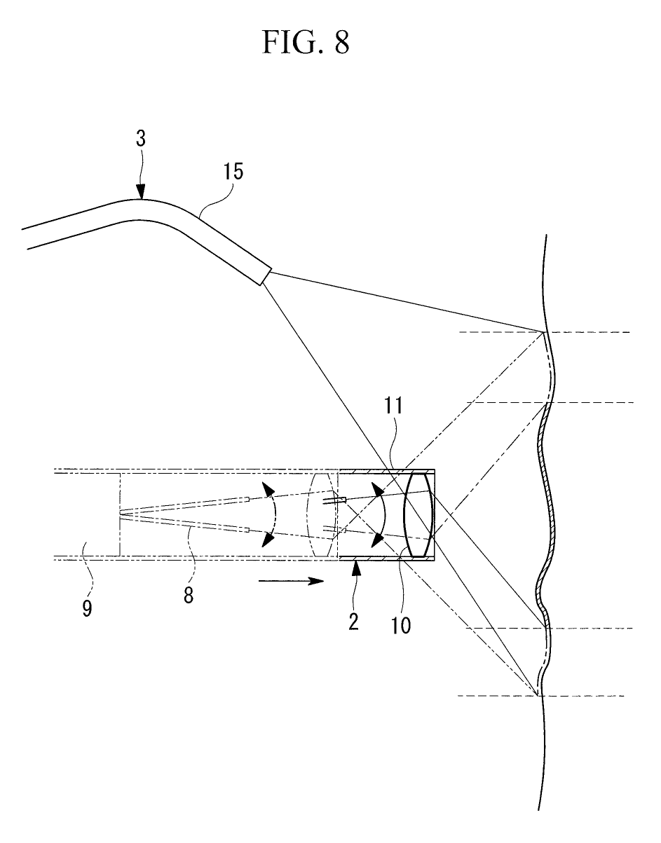

Furthermore, in a case where a microstructure of the subject is to be observed, the output end of the first insertion section 2 is moved close to the subject, as shown in FIG. 8. In this case, since the second insertion section 3 does not need to be moved close to the subject simultaneously with the first insertion section 2, reflection light with excessive intensity does not have to be input to the light-receiving end of the optical fiber 14 included in the second insertion section 3. This is advantageous in that the occurrence of halation in the image can be suppressed.

A known observation system, such as a charge-coupled device (CCD) camera, equipped with two insertion sections similar to those in the present invention includes an illumination unit that serves as one of the insertion sections and that illuminates a subject, and also includes an imaging unit (detection unit) that serves as the other insertion section and that detects reflection light from the subject. When performing observation, such a system prevents the illumination unit from being displaced, radiates the illumination light over a wide region of the subject in the state where the illumination unit is prevented from being displaced, and appropriately sets the imaging unit so that a desired photographic region can be photographed.

In contrast, in the scanning endoscope system 1 according to the present invention, the second insertion section 3 equipped with the light detector 16 is prevented from being displaced so that the reflection light from the subject can be received in a wide region, and observation is performed by appropriately setting the first insertion section 2 that radiates the illumination light onto a desired photographic region in the state where the second insertion section 3 is prevented from being displaced.

The relationship between the illumination unit and the detection unit is inverted between the aforementioned observation system and the scanning endoscope system 1 according to the present invention. If the visual field is to be shifted without changing the position of the subject, in the aforementioned observation system, the user shifts the visual field by moving the imaging unit serving as the detection unit, whereas in the scanning endoscope system 1 according to the present invention, the user shifts the visual field by moving the first insertion section 2 serving as the illumination unit.

As a notable advantage in the scanning endoscope system 1 according to the present invention but not in the aforementioned observation system, when a subject is observed by illuminating a wide region by using the imaging unit, there is a problem of uneven illumination in which the central region is bright but the peripheral region is dark due to the use of diffusion light. In contrast, when a subject is scanned and illuminated by using the scanning endoscope system 1 according to the present invention, the quantity of illumination light does not vary from scan position to scan position, so that uneven illumination can be advantageously prevented.

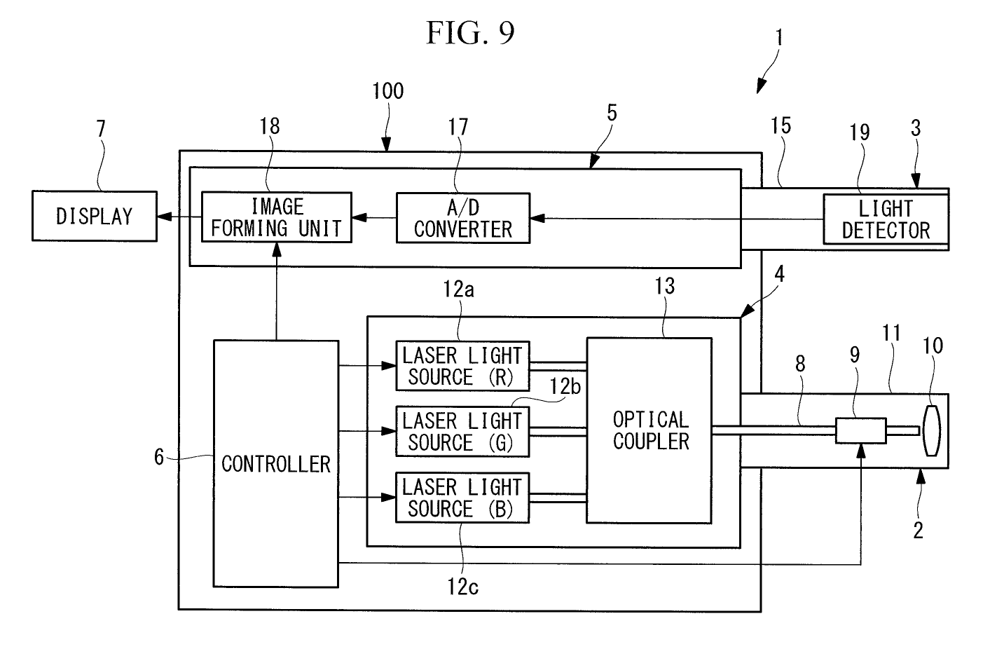

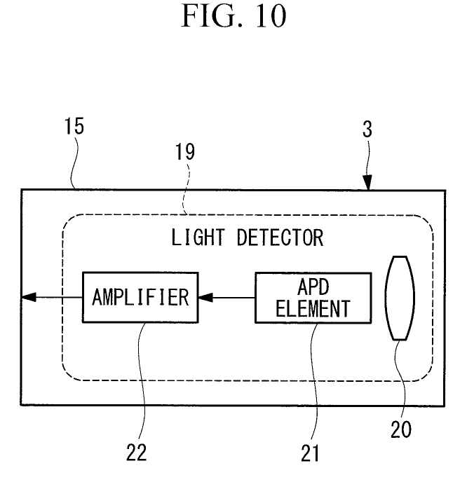

As an alternative to this embodiment in which the second insertion section 3 is equipped with the optical fiber 14 having the light-receiving end that receives the reflection light, a light detector 19 that receives the reflection light without the intervention of the optical fiber 14 may be disposed at the distal end of the second insertion section 3, as shown in FIG. 9. For example, as shown in FIG. 10, the light detector 19 may include a focusing lens 20 that focuses the reflection light, an avalanche photodiode (APD element) 21 that detects the reflection light focused by the focusing lens 20, and an amplifier 22 that amplifies an output of the avalanche photodiode 21.

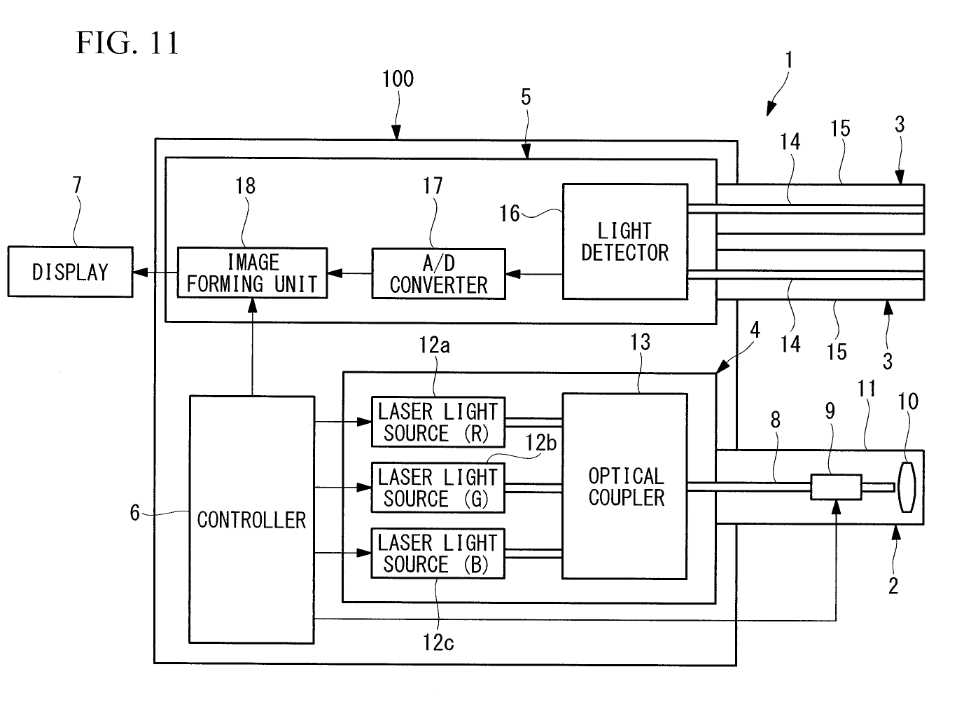

Furthermore, as shown in FIG. 11, a plurality of second insertion sections 3 equipped with optical fibers 14 connected to the light detector 16 may be provided. In this case, the light detector 16 is similar to the light detector 19 in that it includes a focusing lens 20, an APD element 21, and an amplifier 22. Beams of reflection light obtained from the plurality of second insertion sections 3 are optically combined and focused by the focusing lens 20. Thus, a larger quantity of reflection light can be received, and the reflection light from the subject can be received from a plurality of angles, so that a brighter image with no unevenness can be acquired.

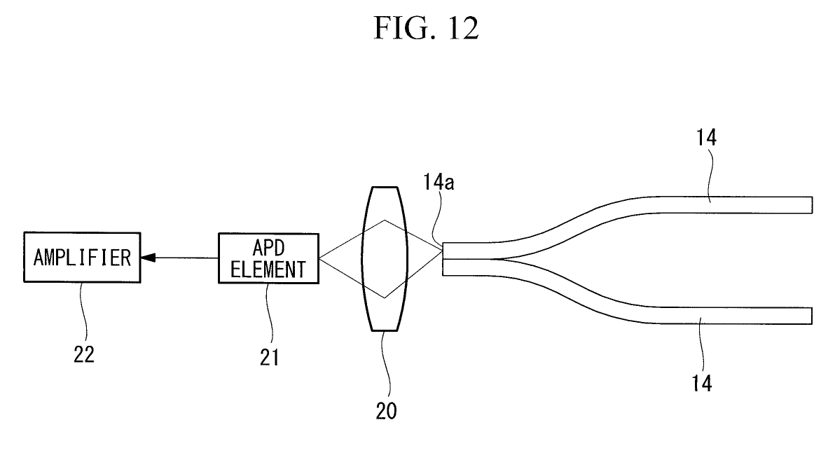

FIG. 12 illustrates an example where light is focused onto the APD element 21 via the optical fibers 14 of the plurality of second insertion sections 3. The output ends of the plurality of optical fibers 14 are bound into a bundle, and light output from an end surface 14a thereof is focused onto a light-receiving surface of the APD element 21 by using the focusing lens 20. With this arrangement, the focusing lens 20 and the APD element 21 can both have a single configuration, so that a complex configuration becomes unnecessary.

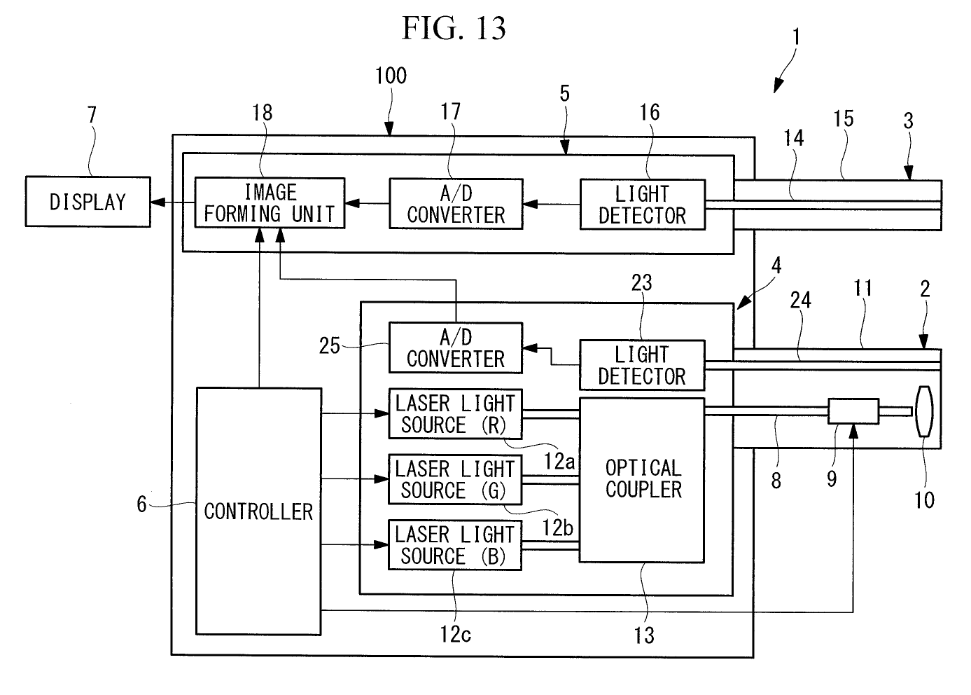

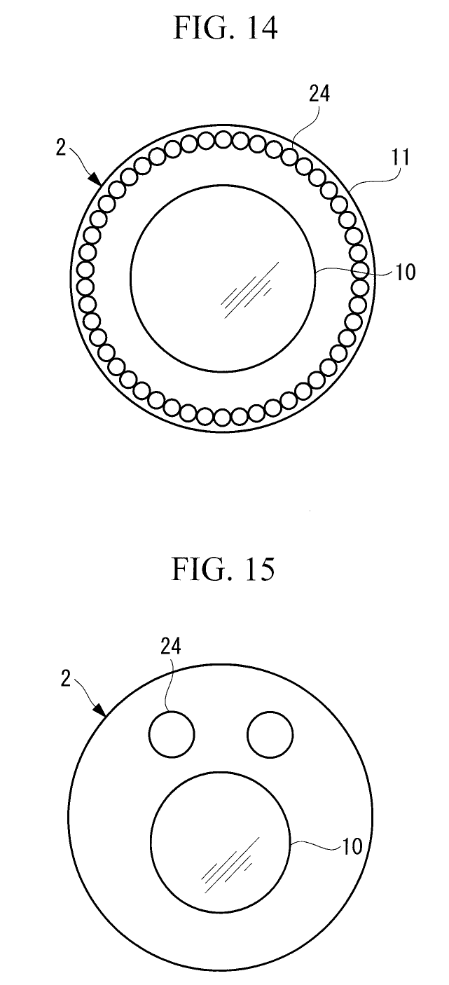

Furthermore, as shown in FIG. 13, in this embodiment, the first insertion section 2 that outputs the illumination light may also be provided with a light detector 23. With regard to the light detector 23 provided in the first insertion section 2, light-receiving ends of optical fibers 24 may be arranged in the form of a ring around the illumination lens 10 that outputs the illumination light, as shown in FIG. 14, or may be arranged next to each other near the illumination lens 10, as shown in FIG. 15. In this case, the light detector 23 connected to the optical fibers 24 of the first insertion section 2 is connected to an A/D converter 25 that converts the detected reflection light into a digital signal.

In the above embodiment, the second insertion section 3 is first inserted into the body, and the first insertion section 2 is subsequently inserted into the body, so that reflection light from the subject, being reflected illumination light emitted from the first insertion section 2, is detected by the second insertion section 3 when the first insertion section 2 is inserted, whereby an image can be displayed. Alternatively, the first insertion section 2 may also be provided with the light detector 23. This is advantageous in that, without having to insert the second insertion section 3, the insertion process can be readily and accurately performed while viewing the image when the first insertion section 2 is inserted.

In this case, since the light detector 23 provided in the first insertion section 2 simply needs to detect a minimum quantity of light necessary for the insertion of the first insertion section 2, the light detector 23 may have a light-receiving area smaller than the light-receiving area of the light-receiving end of the optical fiber 14 provided in the second insertion section 3. Consequently, the first insertion section 2 can be reduced in diameter, which is advantageous in that the invasiveness to the patient can be reduced.

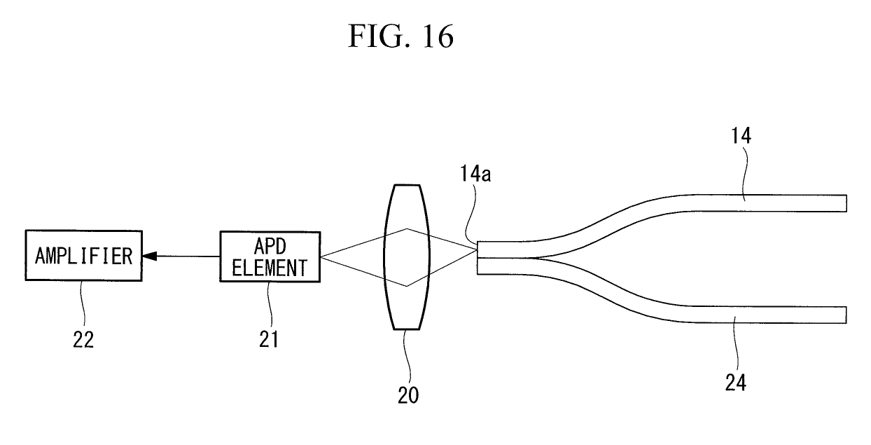

FIG. 16 illustrates an example where the output end of the optical fiber 14 that optically guides light to the second insertion section 3 and the output end of the optical fiber 24 that optically guides light to the light detector 23 are bound into a bundle in this embodiment. Similar to FIG. 12, light output from the bound end surface 14a is focused onto the light-receiving surface of the APD element 21 by using the focusing lens 20. With this arrangement, it is not necessary to configure the plurality of light detectors 16 and 23 and A/D converters 17 and 25 as in the scanning endoscope system 1 shown in FIG. 13, so that the optical system, the APD element, and the A/D converter can have a single configuration, whereby a complex configuration becomes unnecessary.

With regard to the set dimensions of the area of the light-receiving end that receives reflection light from a subject A, for example, if an optical fiber bundle is disposed at the light-receiving end, as described above, the dimensions of the area of the fiber bundle may be adjusted. Alternatively, if a photodiode (PD) is disposed at the light-receiving end, the dimensions of the light-receiving area thereof or the number of PDs may be adjusted.

With regard to the image to be displayed on the display 7, the image of the first insertion section 2 on the display 7 may be changed to the image of the second insertion section 3 when the second insertion section 3 is inserted, two images, namely, the images of the first insertion section 2 and the second insertion section 3, may be displayed on the display 7, or an image obtained by combining signals of the two images may be displayed.

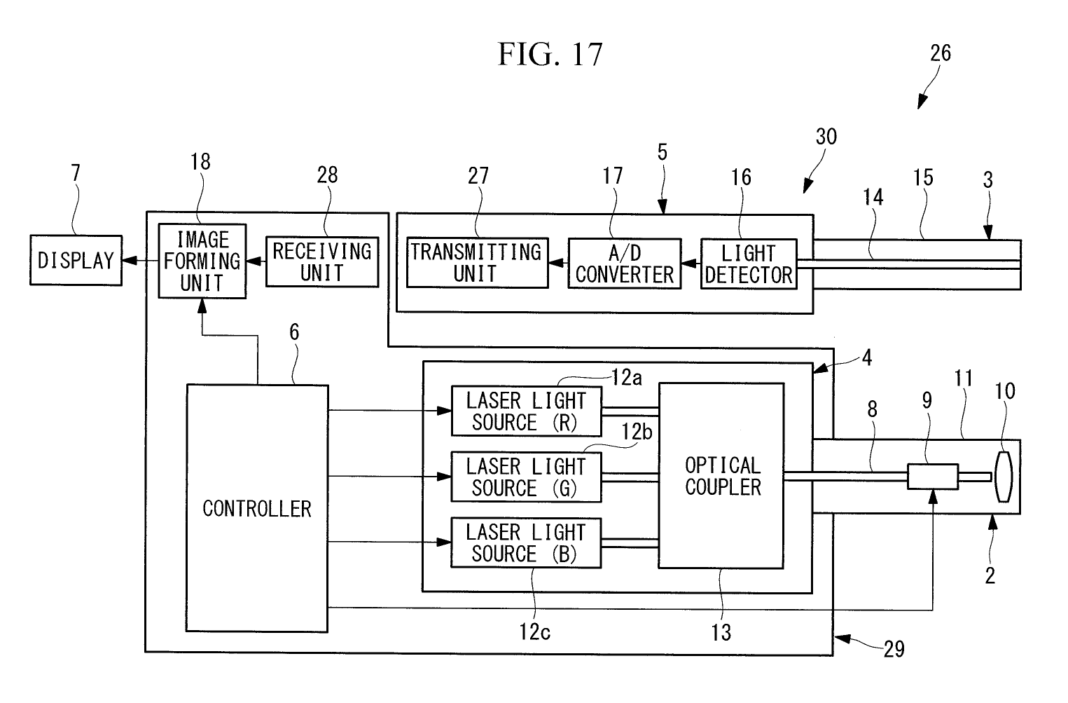

The first insertion section 2 that outputs the illumination light and the second insertion section 3 that detects the reflection light may be connected in a wired manner, or may exchange digital signals in a wireless manner, as shown in FIG. 17.

In detail, a scanning endoscope system 26 that includes: a first endoscope system 29 including the first insertion section 2, the light source unit 4, the controller 6, the image forming unit 18, and a receiving unit 28; a second endoscope system 30 including the second insertion section 3, the light detector 16, the A/D converter 17, and a transmitting unit 27; and the display 7 may be employed.

In the second endoscope system 30 including the second insertion section 3, reflection light output from the subject to the first insertion section 2 is input to the second insertion section 3, the intensity information about the input reflection light is detected by the light detector 16, the detected reflection-light intensity information is converted into a digital signal by the A/D converter 17, and the digital signal is sent to the transmitting unit 27.

The transmitting unit 27 transmits the digital signal sent from the A/D converter 17. Then, the digital signal transmitted by the transmitting unit 27 is received by the receiving unit 28 of the first endoscope system 29 including the first insertion section 2, the received digital signal is sent to the image forming unit 18 where an image is generated, and the generated image is displayed on the display 7.

By connecting the first endoscope system 29 and the second endoscope system 30 in a wireless manner, the dependency on the first endoscope system 29 is eliminated, and the degree of operational freedom can be improved without problems, such as tangled cables.

The transmitting unit 27 and the receiving unit 28 may respectively be disposed at any position in the first endoscope system 29 and the second endoscope system 30.

As an alternative to this embodiment that uses the piezoelectric elements 92a, 92b, 92c, and 92d as the light scanner 9, the method for scanning the illumination light is not limited to this, and an electromagnetic induction method or a galvanometer mirror may be used.

Furthermore, as an alternative to this embodiment in which three kinds of illumination light are sequentially output from the three laser light sources 12a, 12b, and 12c in a predetermined light emission order, for example, light may be output simultaneously from the three laser light sources 12a, 12b, and 12c, and the colors may be separated from one another at the light detector 16, so that the intensity information about each color may be obtained.

In this case, the light detector 16 is constituted of an optical element, such as a dichroic prism, and photoelectric conversion elements, such as avalanche photodiodes, for the respective colors.

Moreover, in this embodiment, the light detector 16 may be provided in the second insertion section 3 instead of the image acquiring unit 5.

The above-described embodiment leads to the following invention.

An aspect of the present invention provides a scanning endoscope system including: an illumination-light emitting unit that outputs illumination light emitted from a light source in a spot-like manner toward a subject; a light scanner that scans the illumination light output from the illumination-light emitting unit over the subject; and a light detector that is provided in a movable manner relative to the illumination-light emitting unit and that detects reflection light from a scan position on the subject over which the illumination light is scanned by the light scanner.

According to this aspect, when the illumination light output from the light source is scanned by the light scanner and is output toward the subject from the illumination-light emitting unit, the reflection light in the subject is received and detected by the light detector. If the scan range in the light scanner exceeds a light-receiving range set in accordance with the numerical aperture of a detection optical fiber, the light detector is moved relatively to the illumination-light emitting unit in a direction away from the subject, so that a photographic field angle corresponding to the illumination range can be achieved.

In this case, a photographic field angle corresponding to a wide illumination range can be achieved by simply moving the light detector without changing the position of the illumination-light emitting unit, so that the illumination-light emitting unit is prevented from being positionally displaced, whereby the spot diameter of the illumination light on the subject can be kept fixed and the resolution can be maintained.

In addition, even if the illumination-light emitting unit is moved close to the subject for observing the microstructure of the subject, the light detector does not need to be simultaneously moved close to the subject, thereby suppressing an increase in detection light quantity and preventing halation from occurring.

In the above aspect, the scanning endoscope system may further include a first insertion section and a second insertion section that are to be inserted into a body of a patient, and a housing to which proximal ends of the first insertion section and the second insertion section are connected and that is disposed outside the body of the patient. The illumination-light emitting unit may be provided in the first insertion section, and the light detector may be provided in the second insertion section.

Accordingly, the illumination-light emitting unit and the light detector are separately provided in the first insertion section and the second insertion section, respectively, thereby reducing the outer diameters of the two insertion sections and further reducing the invasiveness.

Furthermore, in the above aspect, the scanning endoscope system may further include a first insertion section and a second insertion section that are to be inserted into a body of a patient, and a housing to which proximal ends of the first insertion section and the second insertion section are connected and that is disposed outside the body of the patient. The illumination-light emitting unit may be provided in the first insertion section, and the light detector may be provided in the housing and may detect the reflection light received by the second insertion section.

Moreover, in the above aspect, the second insertion section may include a plurality of the second insertion sections.

Accordingly, with the plurality of light detectors provided in the plurality of second insertion sections, a larger quantity of reflection light from the subject can be detected, so that a bright image can be acquired.

Furthermore, in the above aspect, the light detector may include a plurality of the light detectors, and the first insertion section may include at least one of the light detectors.

Accordingly, when the first insertion section is inserted into the body in a state where the light detector provided in the second insertion section is not inserted in the body, a portion of the reflection light, from the subject, of the illumination light output from the illumination-light emitting unit of the first insertion section is detected by the light detector provided in the first insertion section. Consequently, an image of the subject can be formed based on the reflection light detected by the light detector provided in the first insertion section, so that the operator can readily and properly perform the process for inserting the illumination-light emitting unit while viewing the image.

Furthermore, in the above aspect, the light detector may include a plurality of the light detectors, and the reflection light received by the first insertion section may be detected by at least one of the light detectors provided in the housing.

Moreover, in the above aspect, a light-receiving area for the reflection light received by the first insertion section may be smaller than a light-receiving area for the reflection light received by the second insertion section.

Accordingly, an increase of the outer diameter of the illumination-light emitting unit by the light detector provided in the first insertion section can be minimized, thereby reducing the invasiveness when inserting the illumination-light emitting unit into the body.

Furthermore, in the above aspect, the scanning endoscope system may further include: a second endoscope system including the second insertion section and a transmitting unit that transmits a signal based on the reflection light detected by the light detector of the second insertion section; and a first endoscope system including the first insertion section, a receiving unit that receives the signal transmitted by the transmitting unit, and an image forming unit that forms an image from the signal received by the receiving unit.

Accordingly, intensity information about the reflection light in the subject detected by the light detector of the second insertion section is transmitted in the form of a signal from the second endoscope system to the first endoscope system by the transmitting unit. Then, the receiving unit of the first endoscope system receives the signal from the transmitting unit of the second endoscope system, and the received signal is input to the image forming unit where an image is generated. Specifically, the first and second endoscope systems are connected to each other in a wireless manner, thereby eliminating the dependency on the first endoscope system and further improving the degree of operational freedom without problems, such as tangled cables.

### REFERENCE SIGNS LIST

1, 26 scanning endoscope system

2 first insertion section (illumination-light emitting unit)

3 second insertion section (light detector)

4 light source unit (light source)

9 light scanner

18 image forming unit

23 light detector

27 transmitting unit

28 receiving unit

29 first endoscope system

30 second endoscope system

100 housing

1. A scanning endoscope system comprising:
an illumination-light emitting unit that outputs illumination light emitted from a light source in a spot-like manner toward a subject;
a light scanner that scans the illumination light output from the illumination-light emitting unit over the subject; and
a light detector that is provided in a movable manner relative to the illumination-light emitting unit and that detects reflection light from a scan position on the subject over which the illumination light is scanned by the light scanner.

2. The scanning endoscope system according to claim 1, further comprising:
a first insertion section and a second insertion section that are to be inserted into a body of a patient; and
a housing to which proximal ends of the first insertion section and the second insertion section are connected and that is disposed outside the body of the patient,
wherein the illumination-light emitting unit is provided in the first insertion section, and
wherein the light detector is provided in the second insertion section.

3. The scanning endoscope system according to claim 1, further comprising:
a first insertion section and a second insertion section that are to be inserted into a body of a patient; and
a housing to which proximal ends of the first insertion section and the second insertion section are connected and that is disposed outside the body of the patient,
wherein the illumination-light emitting unit is provided in the first insertion section, and
wherein the light detector is provided in the housing and detects the reflection light received by the second insertion section.

4. The scanning endoscope system according to claim 2,
wherein the second insertion section includes a plurality of the second insertion sections.

5. The scanning endoscope system according to claim 3,
wherein the second insertion section includes a plurality of the second insertion sections.

6. The scanning endoscope system according to claim 4,
wherein the light detector includes a plurality of the light detectors, and
wherein the first insertion section includes at least one of the light detectors.

7. The scanning endoscope system according to claim 5,
wherein the light detector includes a plurality of the light detectors, and
wherein the first insertion section includes at least one of the light detectors.

8. The scanning endoscope system according to claim 3,
wherein the light detector includes a plurality of the light detectors, and
wherein the first insertion section includes at least one of the light detectors.

9. The scanning endoscope system according to claim 4,
wherein the light detector includes a plurality of the light detectors, and
wherein the reflection light received by the first insertion section is detected by at least one of the light detectors provided in the housing.

10. The scanning endoscope system according to claim 5,
wherein the light detector includes a plurality of the light detectors, and
wherein the reflection light received by the first insertion section is detected by at least one of the light detectors provided in the housing.

11. The scanning endoscope system according to claim 3,
wherein the light detector includes a plurality of the light detectors, and
wherein the reflection light received by the first insertion section is detected by at least one of the light detectors provided in the housing.

12. The scanning endoscope system according to claim 6,
wherein a light-receiving area for the reflection light received by the first insertion section is smaller than a light-receiving area for the reflection light received by the second insertion section.

13. The scanning endoscope system according to claim 9,
wherein a light-receiving area for the reflection light received by the first insertion section is smaller than a light-receiving area for the reflection light received by the second insertion section.

14. The scanning endoscope system according to claim 7,
wherein a light-receiving area for the reflection light received by the first insertion section is smaller than a light-receiving area for the reflection light received by the second insertion section.

15. The scanning endoscope system according to claim 10,
wherein a light-receiving area for the reflection light received by the first insertion section is smaller than a light-receiving area for the reflection light received by the second insertion section.

16. The scanning endoscope system according to claim 8,
wherein a light-receiving area for the reflection light received by the first insertion section is smaller than a light-receiving area for the reflection light received by the second insertion section.

17. The scanning endoscope system according to claim 11,
wherein a light-receiving area for the reflection light received by the first insertion section is smaller than a light-receiving area for the reflection light received by the second insertion section.

18. The scanning endoscope system according to claim 4, further comprising:
a second endoscope system including the second insertion section and a transmitting unit that transmits a signal based on the reflection light detected by the light detector of the second insertion section; and
a first endoscope system including the first insertion section, a receiving unit that receives the signal transmitted by the transmitting unit, and an image forming unit that forms an image from the signal received by the receiving unit.

19. The scanning endoscope system according to claim 2, further comprising:
a second endoscope system including the second insertion section and a transmitting unit that transmits a signal based on the reflection light detected by the light detector of the second insertion section; and
a first endoscope system including the first insertion section, a receiving unit that receives the signal transmitted by the transmitting unit, and an image forming unit that forms an image from the signal received by the receiving unit.

20. The scanning endoscope system according to claim 3, further comprising:
a second endoscope system including the second insertion section and a transmitting unit that transmits a signal based on the reflection light detected by the light detector of the second insertion section; and
a first endoscope system including the first insertion section, a receiving unit that receives the signal transmitted by the transmitting unit, and an image forming unit that forms an image from the signal received by the receiving unit.
